(19) United States
(12) Patent Application Publication
(10) Pub. No.: US 2025/0308029 A1
(43) Pub. Date: Oct. 2, 2025

(54) ANALYSIS APPARATUS, ULTRASOUND DIAGNOSTIC APPARATUS, AND NON-TRANSITORY COMPUTER-READABLE STORAGE MEDIUM

(71) Applicant: Canon Medical Systems Corporation, Otawara-shi (JP)

(72) Inventor: Koji ANDO, Otawara (JP)

(73) Assignee: Canon Medical Systems Corporation, Otawara-shi (JP)

(21) Appl. No.: 19/087,829

(22) Filed: Mar. 24, 2025

(30) Foreign Application Priority Data
Mar. 27, 2024 (JP) 2024-051790
Mar. 18, 2025 (JP) 2025-043922

(51) Int. Cl. G06T 7/00; A61B 8/08

(52) U.S. Cl. CPC G06T 7/0016; A61B 8/0883; G06T 2200/24; G06T 2207/10132; G06T 2207/20092; G06T 2207/30048

(57) ABSTRACT

According to one embodiment, an analysis apparatus includes processing circuitry. The processing circuitry obtains a thumbnail of each of multiple pieces of cross-section image data related to first cross-section image data selected by a user and showing a cross-section of an organ, specifies, using the multiple thumbnails obtained, a cross-section of an organ included in each of the multiple thumbnails, and selects a cross-section for use as a target of analysis other than a cross-section selected by the user from the specified cross-sections based on a degree of association between the first cross-section image data and each of the multiple pieces of cross-section image data.

### CROSS-REFERENCE TO RELATED APPLICATIONS

This application is based upon and claims the benefit of priority from Japanese Patent Applications No. 2024-051790, filed Mar. 27, 2024; and No. 2025-43922, filed Mar. 18, 2025; the entire contents of all of which are incorporated herein by reference.

### FIELD

Embodiments described herein relate generally to an analysis apparatus, an ultrasound diagnostic apparatus, and a non-transitory computer-readable storage medium.

### BACKGROUND

Conventionally, when multiple cross-sections of an organ such as a heart used for a desired analysis such as a myocardial analysis are to be acquired using an ultrasound probe, a function called “an acquisition assisting function” is available, in which cross-sections necessary for the myocardial analysis are automatically acquired by designating for a user the order of the cross-sections to be acquired. There is also a function called “an AI obtaining function”, in which the types of cross-sections are estimated using AI (artificial intelligence) for multiple cross-sections of a heart acquired by a user and cross-sections necessary for a myocardial analysis are automatically obtained.

However, these functions have the following challenges. The acquisition assisting function, for example, does not allow acquisition of cross-sections in an order optimal for a user, causing a burden on the user. Also, the AI obtaining function requires, when obtaining necessary cross-sections, generation of images from DICOM (digital imaging and communication in medicine) data and analysis of the images for all the cross-sections, involving image generation and analysis for unnecessary cross-sections as well, thus requiring time. Consequently, the conventional functions sometimes take time when selecting cross-sections suitable for the analysis. Therefore, a technology that makes it possible to efficiently select cross-sections suitable for a desired analysis is needed.

### BRIEF DESCRIPTION OF THE DRAWINGS

FIG. 1 is a block diagram showing an example of a configuration of an ultrasound diagnostic apparatus according to a first embodiment.

FIG. 2 is a flowchart for explaining an operation of processing circuitry that performs an analysis-target extraction process according to the first embodiment.

FIG. 3 is a flowchart illustrating a user-selected-cross-section process shown in FIG. 2.

FIG. 4 is a flowchart illustrating an another-cross-section selecting process shown in FIG. 2.

FIG. 5 is a flowchart illustrating a selected-cross-section determining process shown in FIG. 2.

FIG. 6 is a diagram for illustrating a display screen of a myocardial function analysis application according to the first embodiment.

FIG. 7 is a diagram for explaining a state of a thumbnail image display region in which thumbnail images as candidates for analysis are displayed in a highlighted manner according to the first embodiment.

FIG. 8 is a diagram for explaining a state of a thumbnail image display region in which an order of analysis is displayed on thumbnail images as candidates for analysis according to the first embodiment.

FIG. 9 is a diagram for explaining a state of a thumbnail image display region in which the names of cross-sections are displayed on thumbnail images as candidates for analysis according to the first embodiment.

FIG. 10 is a diagram for explaining a state of a thumbnail image display region in which thumbnail images as targets of analysis are displayed in an emphasized manner according to the first embodiment.

FIG. 11 is a diagram for illustrating a display screen posterior to the analysis of the myocardial function analysis application according to the first embodiment.

FIG. 12 is a diagram for explaining a state of a thumbnail image display region in which candidates for analysis are rearranged according to a first modification of the first embodiment.

FIG. 13 is a diagram for explaining a state of a thumbnail image display region in which thumbnail images other than candidates for analysis are displayed as non-selected according to a second modification of the first embodiment.

FIG. 14 is a block diagram showing an example of a configuration of an analysis apparatus according to a second embodiment.

### DETAILED DESCRIPTION

In general, according to one embodiment, an analysis apparatus includes processing circuitry. The processing circuitry obtains a thumbnail of each of multiple pieces of cross-section image data related to first cross-section image data selected by a user and showing a cross-section of an organ, specifies, using the multiple thumbnails obtained, a cross-section of an organ included in each of the multiple thumbnails, and selects a cross-section for use as a target of analysis other than a cross-section selected by the user from the specified cross-sections based on a degree of association between the first cross-section image data and each of the multiple pieces of cross-section image data.

Hereinafter, embodiments of an ultrasound diagnostic apparatus and an analysis apparatus will be described in detail with reference to the accompanying drawings.

### First Embodiment

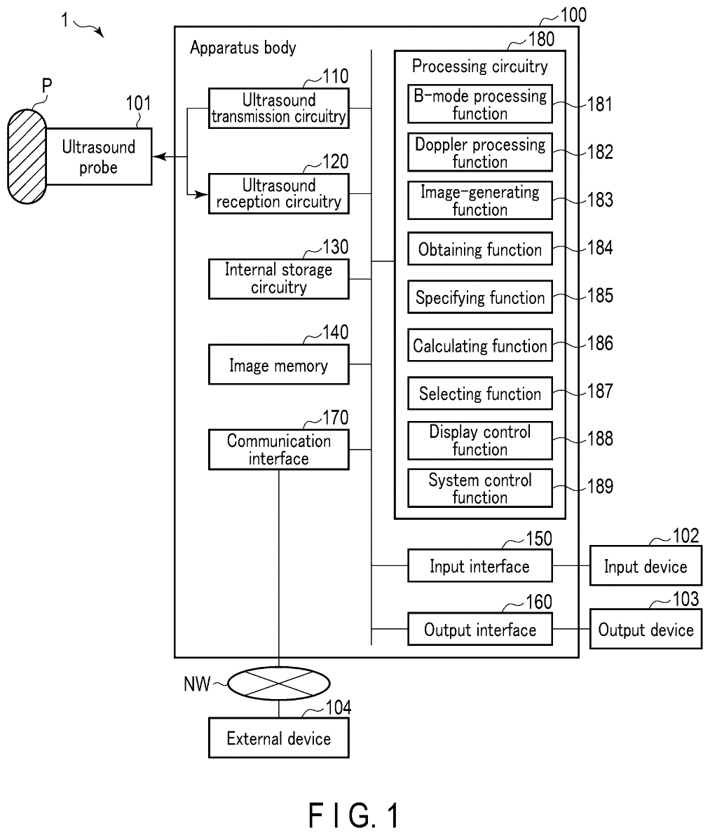

FIG. 1 is a block diagram showing an example of a configuration of an ultrasound diagnostic apparatus 1 according to a first embodiment. The ultrasound diagnostic apparatus 1 shown in FIG. 1 includes an apparatus body 100 and an ultrasound probe 101. The apparatus body 100 is connected to an input device 102 and an output device 103. The apparatus body 100 is also connected to an external device 104 via a network NW. The external device 104 is, for example, a server equipped with a picture archiving and communication system (PACS).

The ultrasound probe 101 performs ultrasonic scanning of a scan area in a living body P, which is a subject, under the control of, for example, the apparatus body 100. The ultrasound probe 101 includes, for example, a plurality of piezoelectric vibrators, a matching layer between a case and the plurality of piezoelectric vibrators, and a backing material that prevents ultrasound waves from propagating backward with respect to a radiation direction from the piezoelectric vibrators. The ultrasound probe 101 is, for example, a sector electronic scanning probe. The ultrasound probe 101 is detachably connected to the apparatus body 100. The ultrasound probe 101 may be provided with buttons that are pressed when an offset process, an operation of freezing an ultrasound image (i.e., freeze operation), and the like are performed.

The piezoelectric vibrators generate ultrasound waves based on a drive signal supplied from ultrasound transmission circuitry 110 (described later) included in the apparatus body 100. Thus, ultrasound waves are transmitted from the ultrasound probe 101 to the living body P. When ultrasound waves are transmitted from the ultrasound probe 101 to the living body P, the transmitted ultrasound waves are sequentially reflected on a surface of a body tissue in the living body P that shows discontinuity in acoustic impedance, and are received by the piezoelectric vibrators as reflected wave signals. The amplitudes of the reflected wave signals received depend on the difference in the acoustic impedance on the surface showing discontinuity on which the ultrasound waves are reflected. If transmitted ultrasound pulses are reflected on a bloodstream or a surface of a cardiac wall or the like in motion, the frequencies of the reflected wave signals are shifted, due to the Doppler effect, according to the velocity components in the ultrasound transmission direction of the moving object. The ultrasound probe 101 receives the reflected wave signals from the living body P, and converts the reflected wave signals into electrical signals.

FIG. 1 illustrates a connection relationship between a single ultrasound probe 101 and the apparatus body 100. However, a plurality of ultrasound probes can be connected to the apparatus body 100. Which of the connected ultrasound probes is to be used for the ultrasound scanning can be selected discretionarily via, for example, a software button on a touch panel described later.

The apparatus body 100 is an apparatus that generates an ultrasound image based on the reflected wave signals received by the ultrasound probe 101. The apparatus body 100 includes ultrasound transmission circuitry 110, ultrasound reception circuitry 120, internal storage circuitry 130, an image memory 140, an input interface 150, an output interface 160, a communication interface 170, and processing circuitry 180.

The ultrasound transmission circuitry 110 is a processor that supplies a drive signal to the ultrasound probe 101. The ultrasound transmission circuitry 110 is realized by, for example, trigger generation circuitry, delay circuitry, and pulser circuitry. The trigger generation circuitry repeatedly generates rate pulses for forming transmission ultrasound waves at a predetermined rate frequency. The delay circuitry gives each rate pulse generated by the trigger generation circuitry a delay time for each piezoelectric vibrator needed to converge the ultrasound waves generated from the ultrasound probe into a beam and determine the transmission directivity. The pulser circuitry applies drive signals (drive pulses) to a plurality of ultrasound vibrators provided in the ultrasound probe 101 at a timing based on the rate pulse. The transmission direction from the surfaces of the piezoelectric vibrators can be discretionarily adjusted by varying the delay time given to each rate pulse by the delay circuitry.

The ultrasound transmission circuitry 110 can discretionarily change the output intensity of the ultrasound waves through a drive signal. In the ultrasound diagnostic apparatus, the influence of the attenuation of the ultrasound waves in the living body P can be reduced by increasing the output intensity. By reducing the influence of the attenuation of the ultrasound waves, the ultrasound diagnostic apparatus can obtain a reflected wave signal having a large S/N ratio when receiving the signal.

In general, when the ultrasound waves are propagated inside the living body P, the strength of the vibration of the ultrasound waves corresponding to the output intensity (the strength is also referred to as “acoustic power”) is attenuated. The attenuation of the acoustic power is caused by absorption, scattering, reflection, and the like. The degree of reduction of the acoustic power depends on the frequency of the ultrasound waves and the distance of the ultrasound waves in the radial direction. For example, the degree of attenuation is increased by increasing the frequency of the ultrasound waves. The longer the distance of the ultrasound waves in the radial direction, the larger the degree of attenuation.

The ultrasound reception circuitry 120 is a processor that performs various types of processing on the reflected wave signals received by the ultrasound probe 101 and generates reception signals. The ultrasound reception circuitry 120 generates reception signals based on the reflected wave signals of the ultrasound waves obtained by the ultrasound probe 101. Specifically, the ultrasound reception circuitry 120 is realized by, for example, a preamplifier, an A/D converter, a demodulator, and a beam former. The preamplifier performs gain correction by amplifying, for each channel, the reflected wave signals received by the ultrasound probe 101. The A/D converter converts the gain-corrected reflected wave signals into digital signals. The demodulator demodulates the digital signals. The beam former, for example, gives the demodulated digital signals a delay time needed to determine the reception directivity, and adds a plurality of digital signals given the delay time. Through the addition performed by the beam former, reception signals with an enhanced reflection component from a direction corresponding to the reception directivity are generated.

The internal storage circuitry 130 includes, for example, a processor-readable storage medium such as a magnetic storage medium, an optical storage medium, or a semiconductor memory. The internal storage circuitry 130 stores a program for implementing ultrasound transmission-reception, a program related to myocardial function analysis (described later), various kinds of data, and the like. The various kinds of data include, for example, parameters and lookup tables (LUTs) that are used during execution of a program. The programs and various kinds of data may be pre-stored in, for example, the internal storage circuitry 130. For example, the programs and various kinds of data may be stored in a non-transitory storage medium, distributed, read from the non-transitory storage medium, and installed in the internal storage circuitry 130. According to an operation that is input via the input interface 150, the internal storage circuitry 130 stores, for example, in DICOM form, B-mode image data, contrast image data, ultrasound image data related to a bloodstream image, and the like that are generated by the processing circuitry 180. For example, the ultrasound image data stored in DICOM form may be referred to as medical image data. The medical image data may be, for example, moving image data. Various pieces of information may be attached to the medical image data as supplementary information. The internal storage circuitry 130 can also transfer the stored image data or moving image data to the external device 104, etc., via the communication interface 170.

The internal storage circuitry 130 may be a drive device or the like which reads and writes various kinds of information to and from a portable storage medium such as a CD drive, a DVD drive, and a flash memory. The internal storage circuitry 130 can also write the stored data into a portable storage medium, and store the data in the external device 104 via the portable storage medium.

The image memory 140 includes, for example, a processor-readable storage medium such as a magnetic storage medium, an optical storage medium, or a semiconductor memory. The image memory 140 stores image data corresponding to multiple frames immediately preceding a freeze operation that is input via the input interface 150. The image data stored in the image memory 140 is, for example, successively displayed (cine-displayed).

The internal storage circuitry 130 and the image memory 140 need not necessarily be realized by independent storage devices. The internal storage circuitry 130 and the image memory 140 may be realized by a single storage device. The internal storage circuitry 130 and the image memory 140 may each be realized by a plurality of storage devices.

The input interface 150 receives various instructions from an operator (user) via the input device 102 (input unit). The input device 102 is, for example, a mouse, a keyboard, a panel switch, a slider switch, a trackball, a rotary encoder, an operation panel, or a touch panel. The input interface 150 is connected to the processing circuitry 180 via, for example, a bus, converts an operation instruction that is input by a user into an electrical signal, and outputs the electrical signal to the processing circuitry 180. The input interface 150 is not limited to a component that is connected to physical operational components such as a mouse and a keyboard. Examples of the input interface also include circuitry that receives an electrical signal corresponding to an operation instruction that is input from an external input device provided separately from the ultrasound diagnostic apparatus 1 and outputs the electrical signal to the processing circuitry 180.

The output interface 160 is, for example, an interface for outputting an electrical signal from the processing circuitry 180 to the output device 103. The output device 103 is any display such as a liquid crystal display, an organic EL display, an LED display, a plasma display, or a CRT display. The output device 103 may be a touch-panel display that also serves as the input device 102. The output device 103 may further include a speaker that outputs voice in addition to a display. The output interface 160 is connected to the processing circuitry 180 via a bus, for example, and outputs an electrical signal from the processing circuitry 180 to the output device 103.

The communication interface 170 is connected to the external device 104 via a network NW, for example, and performs data communication with the external device 104.

The processing circuitry 180 is, for example, a processor that functions as the center of the ultrasound diagnostic apparatus 1. The processing circuitry 180 executes a program stored in the internal storage circuitry 130 (storage), thereby implementing a function corresponding to the program. The processing circuitry 180 includes, for example, a B-mode processing function 181, a Doppler processing function 182, an image-generating function 183, an obtaining function 184 (obtaining unit), a specifying function 185 (specifying unit), a calculating function 186 (calculating unit), a selecting function 187 (selecting unit), a display control function 188 (display controller), and a system control function 189 (controller).

The B-mode processing function 181 is a function of generating B-mode data based on the reception signals (echo signals) received from the ultrasound reception circuitry 120. In the B-mode processing function 181, the processing circuitry 180, for example, performs an envelope detection process, a logarithmic compression process, and the like on the reception signals received from the ultrasound reception circuitry 120 to generate data (B-mode data) that represents the signal intensity of the reception signals (echo reflection intensity) with a value of brightness (brightness value). The generated B-mode data is stored in a RAW data memory (not shown) as B-mode RAW data on a two-dimensional ultrasound scanning line (raster).

Also, the processing circuitry 180 can perform harmonic imaging with the B-mode processing function 181. The harmonic imaging is an imaging method that uses not only a fundamental wave component but also a harmonic wave component (harmonic component) that is included in the reflection wave signals of the ultrasound waves. The harmonic imaging includes, for example, a tissue harmonic imaging (THI) that does not use a contrast agent and a contrast harmonic imaging (CHI) that uses a contrast agent.

In the THI, a harmonic component can be extracted using an amplitude modulation (AM) method, a phase modulation (PM) method, or an imaging method called an AMPM method, which is a combination of the AM method and the PM method.

In the AM method, the PM method, and the AMPM method, ultrasound transmission is performed more than once for a single scanning line, with varying amplitudes and/or phases. Through this process, the ultrasound reception circuitry 120 generates multiple pieces of reflection wave data at each scanning line, and outputs the generated reflection wave data. With the B-mode processing function 181, the processing circuitry 180 extracts a harmonic component by performing addition-subtraction processing on the multiple pieces of reflection wave data at each scanning line according to a modulation method. The processing circuitry 180 then performs envelope detection processing or the like on the reflection wave data of the harmonic component to generate B-mode data.

For example, in the CHI, a harmonic component is extracted using a frequency filter. With the B-mode processing function 181, the processing circuitry 180 can separate reflected wave data (a harmonic wave component) whose reflection source is a contrast agent from reflected wave data (a fundamental wave component) whose reflection source is a tissue in the living body P. Thus, the processing circuitry 180 can select the harmonic wave component from the contrast agent using a filter and generate B-mode data for generating contrast image data.

The B-mode data for generating contrast image data is data representing, by a brightness value, the intensity of the echo reflection whose reflection source is a contrast agent. The processing circuitry 180 can also extract a fundamental wave component from the reflection wave data of the living body P and generate B-mode data for generating tissue image data.

The Doppler processing function 182 is a function of generating data (Doppler information) of extracted Doppler effect-based motion information of a moving object in a region of interest (ROI) set in a scan area by analyzing the frequencies of the reception signals received from the ultrasound reception circuitry 120. The generated Doppler information is stored in a RAW data memory (not shown) as Doppler RAW data (also referred to as “Doppler data”) on a two-dimensional ultrasound scanning line.

Specifically, with the Doppler processing function 182, the processing circuitry 180 estimates, for example, an average velocity, an average variance value, an average power value, etc., at each sampling point as motion information of a moving object, and generates Doppler data showing the estimated motion information. The moving objects are, for example, a bloodstream, tissues of a cardiac wall and the like, and a contrast agent. With the Doppler processing function 182, the processing circuitry 180 according to the present embodiment estimates, at each sampling point, an average bloodstream velocity, a variance value of a bloodstream velocity, a power value of a bloodstream signal, etc., as bloodstream motion information (bloodstream information), and generates Doppler data showing the estimated bloodstream information.

The image-generating function 183 is a function to generate B-mode image data based on the data generated by the B-mode processing function 181. With the image-generating function 183, the processing circuitry 180, for example, converts (scan-converts) a scanning line signal sequence of ultrasound scanning into a scanning line signal sequence in a video format representatively used by a television, etc., and generates image data for display (display image data). Specifically, the processing circuitry 180 generates two-dimensional B-mode image data (also referred to as “ultrasound image data”) constituted by pixels by subjecting B-mode RAW data stored in the RAW data memory to RAW-pixel conversion such as coordinate conversion according to the mode of the ultrasound scanning performed by the ultrasound probe 101. In other words, with the image-generating function 183, the processing circuitry 180 generates multiple ultrasound images (medical images) corresponding to respective consecutive frames through ultrasound transmission and reception.

The processing circuitry 180 also generates Doppler image data of visualized bloodstream information, for example, by performing RAW-pixel conversion on the Doppler RAW data stored in the RAW data memory. The Doppler image data is average velocity image data, variance image data, power image data, or image data combining any of these. The processing circuitry 180 generates, as the Doppler image data, color Doppler image data showing bloodstream information in color and Doppler image data showing a piece of bloodstream information in waveform on a gray scale.

For example, the processing circuitry 180 may generate a thumbnail image for a user to identify medical image data on a graphical user interface (GUI) by using the medical image data stored in the internal storage circuitry 130. For example, the processing circuitry 180 may automatically generate a thumbnail image at the time when the medical image data is stored in the internal storage circuitry 130. For example, the processing circuitry 180 may generate a preview image having a higher resolution than the resolution of a thumbnail image by using medical image data. For example, the processing circuitry 180 may generate a medical image having a higher resolution than the resolution of a preview image by using medical image data. In the present embodiment, the thumbnail image and the preview image may be referred to as a “low-resolution image” or a “thumbnail image,” and the medical image may be referred to as a “high-resolution image.”

If the medical image data shows a cross-section of an organ, a thumbnail of the medical image data may provide a display such that it is visually and analytically identifiable that a cross-section of an organ is shown. Here, visually identifiable means, for example, that a user having medical knowledge such as a doctor or a technician can identify that the content of the thumbnail shows a cross-section of an organ by visually recognizing the thumbnail. Also, analytically identifiable means, for example, that the processing circuitry 180 can identify that the content of the thumbnail shows a cross-section of an organ by analyzing the thumbnail. In other words, the thumbnail is information regarding a cross-section of an organ. Specifically, if the medical image data is moving image data, the thumbnail may be generated based on the first frame of the moving image data. The medical image data showing a cross-section of an organ may be referred to as “cross-section image data.”

The obtaining function 184 is a function to obtain medical image data (or a low-resolution image of medical image data) necessary for performing a myocardial function analysis process. For example, with the obtaining function 184, the processing circuitry 180 obtains multiple pieces of medical image data related to first medical image data selected by a user or low-resolution images of the respective pieces of medical image data. The low-resolution images are attached to medical image data, for example, as supplementary information. The low-resolution images are, for example, thumbnail images or preview images.

More specifically, as the multiple pieces of medical image data related to the first medical image data or the low-resolution images of the respective pieces of medical image data, the processing circuitry 180 may obtain multiple pieces of medical image data acquired in the same mode as the mode of the ultrasound diagnostic apparatus that acquires the first medical image data or multiple low-resolution images of the multiple pieces of medical image data. The modes of the ultrasound diagnostic apparatus are, for example, a “2D single” mode in which to observe cross-sections, a “4D” mode in which to observe 3D images in motion, a “Doppler” mode in which to observe the flow of a bloodstream, and the like.

The specifying function 185 is a function to specify a cross-section of an organ from an image relating to medical image data. With the specifying function 185, the processing circuitry 180, for example, specifies a cross-section of an organ included in each of the low-resolution images using the obtained low-resolution images. In the case of a heart, the cross-sections are, for example, an apical two chamber view (Apical-2Ch: A2C), an apical three chamber view (Apical-3Ch: A3C), an apical four chamber view (Apical-4Ch: A4C), a parasternal long-axis view (parasternal long-axis: LAx), a parasternal short-axis view (parasternal short-axis: SAx), and the like. For example, a known image recognition technique may be used to specify a cross-section of an organ.

The processing circuitry 180 may specify a first cross-section included in a first low-resolution image of the first medical image data selected by a user. The processing circuitry 180 may specify multiple cross-sections of multiple low-resolution images in order of closeness, i.e., order from the closest to the farthest, of the time at which each of the pieces of medical image data is acquired to the time at which the first medical image data is acquired. Information on the time of acquisition is attached to medical image data, for example, as supplementary information.

The processing circuitry 180 may specify a cross-section of an organ included in each of the high-resolution images using multiple high-resolution images corresponding to multiple low-resolution images.

The calculating function 186 is a function to calculate a degree of association between the first medical image data and each of the multiple pieces of medical image data. For example, with the calculating function 186, the processing circuitry 180 calculates the degree of association based on the time at which the first medical image data is acquired and the time at which each of the multiple pieces of medical image data is acquired. For example, the degree of association based on the time of acquisition increases as the difference between the time at which the first medical image data is acquired and the time at which other medical image data is acquired decreases.

In addition to the time of acquisition, the processing circuitry 180 may calculate the degree of association based on the heart rate and the type of probe (e.g., probe ID) attached to the medical image data. For example, the degree of association based on the heart rate increases as the difference between the heart rate of the first medical image data and the heart rate of other medical image data decreases. For example, the degree of association based on the type of probe increases if the type of probe with which to acquire the first medical image data and the type of probe with which to acquire other medical image data are the same.

In this manner, the calculation of the degree of association may be set appropriately such that the value of the degree of association between the first medical image data selected by a user and medical image data as a target of analysis used in a myocardial function analysis process increases.

The selecting function 187 is a function to select a cross-section used as a target of analysis among the specified cross-sections. For example, with the selecting function 187, the processing circuitry 180 selects another cross-section belonging to the target of analysis including the first cross-section included in the first low-resolution image of the first medical image data from among the specified cross-sections based on the degree of association. The target of analysis is, for example, a group of cross-sections of a heart. In the case of performing a myocardial function analysis process for determining a global longitudinal strain (GLS), the group of heart cross-sections consist of an apical two chamber view, an apical three chamber view, and an apical four chamber view.

The display control function 188 is a function to cause a display as the output device 103 to display images based on various kinds of ultrasound image data generated by the image-generating function 183. Specifically, with the display control function 188, the processing circuitry 180, for example, controls the displaying, on the display, of an image that is based on the B-mode image data, the Doppler image data, or image data including both of the aforementioned types of data that are generated by the image-generating function 183. The processing circuitry 180 may cause a medical image display region for displaying an ultrasound image and a thumbnail image display region for displaying a thumbnail image of medical image data to be displayed.

More specifically, with the display control function 188, the processing circuitry 180, for example, converts (scan-converts) a scanning line signal sequence of ultrasound scanning into a scanning line signal sequence in a video format representatively used by a television, etc., and generates display image data. The processing circuitry 180 may perform various types of processing, such as corrections of a dynamic range, brightness, contrast, and y curve and an RGB conversion, on the display image data. The processing circuitry 180 may add information, such as textual information of various parameters, a scale, a body mark, etc., to the display image data. The processing circuitry 180 may generate a user interface (graphical user interface (GUI)) for an operator to input various instructions through the input device, and cause the GUI to be displayed on the display.

The processing circuitry 180 may cause multiple thumbnail images of the first medical image data and multiple pieces of medical image data to be displayed in the thumbnail image display region in chronological order according to the time at which each piece of medical image data is acquired. The processing circuitry 180 may cause multiple thumbnail images to be displayed such that the thumbnail images are rearranged in order of closeness of the time at which each of the pieces of medical image data is acquired to the time at which the first medical image data is acquired.

The processing circuitry 180 may cause multiple thumbnail images to be displayed such that the display form is changed according to whether they are medical image data related to a target of analysis or not. The medical image data related to a target of analysis is, for example, medical image data necessary for a myocardial function analysis process. Changing the display form is realized by highlighting or emphasizing of a thumbnail image of medical image data necessary for a myocardial function analysis process or a non-selected display of a thumbnail image of medical image data other than the medical image data necessary for a myocardial function analysis process. In the present embodiment, the processing circuitry 180 subjects a thumbnail image as a target of analysis to emphasizing and subjects a thumbnail image as a candidate for analysis to highlighting. The highlighting and the emphasizing may be represented by giving each of the thumbnail images different colors.

The processing circuitry 180 may include an analyzing function (analyzing unit) that performs a myocardial function analysis process. By implementing the analyzing function, the processing circuitry 180 may generate the result of the analysis of the GLS by analyzing a medical image data group related to a target of analysis. The medical image data group includes, for example, medical image data selected by a user.

The system control function 189 is a function to comprehensively control all of the operations of the ultrasound diagnostic apparatus 1. For example, with the system control function 189, the processing circuitry 180 controls the ultrasound transmission circuitry 110 and the ultrasound reception circuitry 120 based on a parameter related to transmission and reception of ultrasound waves.

The above descriptions relate to the configuration of the ultrasound diagnostic apparatus 1 of the first embodiment. Next, an operation of an analysis-target extraction process according to the first embodiment will be described. The analysis-target extraction process according to the first embodiment includes a process on a cross-section selected by a user (a user-selected-cross-section process), a process of selecting another cross-section based on the user-selected cross-section (an another-cross-section selecting process), and a process of determining a selected cross-section (a selected-cross-section determining process).

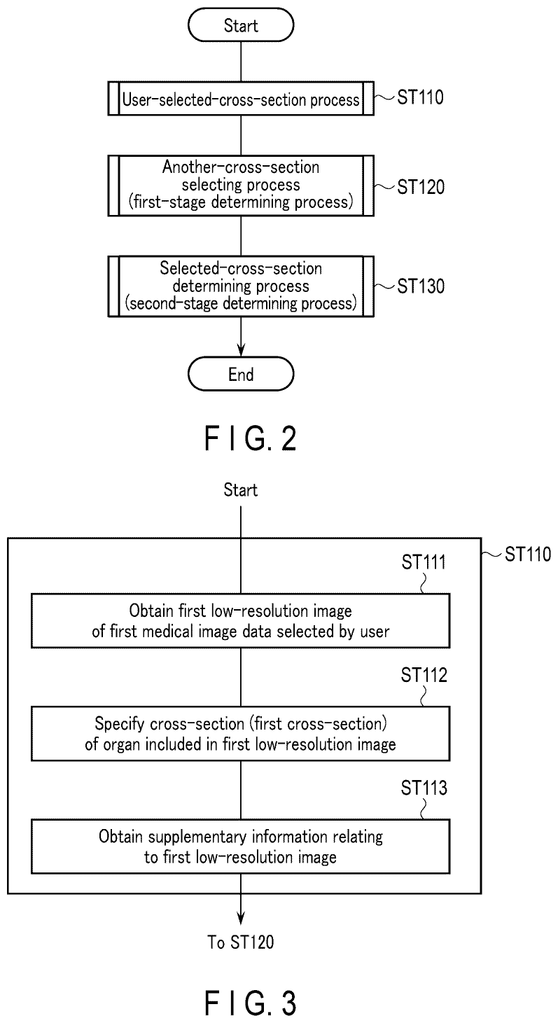

FIG. 2 is a flowchart for explaining an operation of the processing circuitry 180 that performs the analysis-target extraction process according to the first embodiment. The analysis-target extraction process shown in FIG. 2 is started, for example, when a user executes a myocardial function analysis application and selects (or determines) the first medical image data. To give a specific example, a myocardial function analysis application for determining a GLS is executed in the example shown below.

### (Step ST110)

When the myocardial function analysis application is executed and the first medical image data is selected by a user, the processing circuitry 180 performs the user-selected-cross-section process. Hereinafter, a specific example of the user-selected-cross-section process will be explained with reference to the flowchart shown in FIG. 3.

FIG. 3 is a flowchart illustrating the user-selected-cross-section process shown in FIG. 2.

### (Step ST111)

When the processing circuitry 180 performs the user-selected-cross-section process, the processing circuitry 180 implements the obtaining function 184. When the processing circuitry 180 implements the obtaining function 184, the processing circuitry 180 obtains the first low-resolution image of the first medical image data selected by a user. The first low-resolution image is a thumbnail attached to or associated with the first medical image data, and is generated before a selection is made by a user or before the myocardial function analysis application is executed. This holds true of a low-resolution image associated with other medical image data.

### (Step ST112)

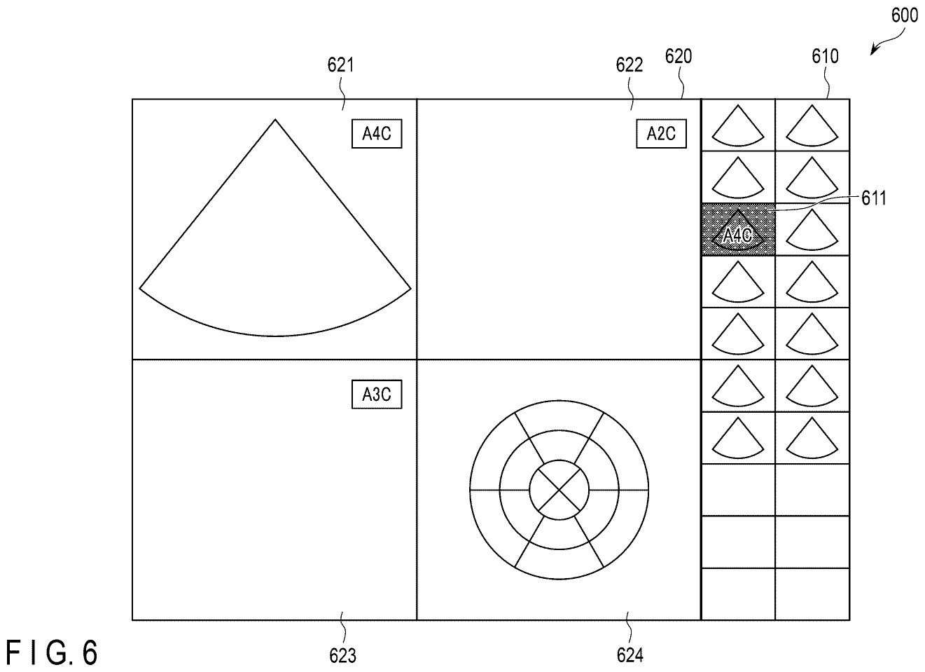

After obtaining the first low-resolution image, the processing circuitry 180 implements the specifying function 185. When the processing circuitry 180 implements the specifying function 185, the processing circuitry 180 specifies a cross-section (first cross-section) of an organ included in the first low-resolution image. A display screen after the first cross-section is specified will be explained below with reference to FIG. 6.

FIG. 6 is a diagram for illustrating a display screen 600 of the myocardial function analysis application according to the first embodiment. The display screen 600 shown in FIG. 6 includes a thumbnail image display region 610 and a medical image display region 620.

Fourteen thumbnail images in a small region of 10 rows×2 columns are displayed in the thumbnail image display region 610. These thumbnail images are displayed in chronological order according to the time at which the medical image data corresponding thereto are acquired. The thumbnail images are arranged in the thumbnail image display region 610 in chronological order in the order of the left side on the first row, the right side on the first row, the left side on the second row, the right side on the second row, continuing likewise. That is, the thumbnail images are arranged such that the medical image data corresponding to the thumbnail image on the upper left side of the thumbnail image display region 610 is the oldest and the medical image data corresponding to the thumbnail image on the lower right side of the thumbnail image display region 610 is the newest.

A user determines a cross-section to be used in the myocardial function analysis by selecting any thumbnail image in the thumbnail image display region 610 and checking a preview screen. The processing circuitry 180 specifies a cross-section (first cross-section) of an organ with regard to the medical image data (first medical image data) corresponding to the thumbnail image selected by the user. In the example shown in FIG. 6, “A4C” is displayed on a thumbnail image 611 since the processing circuitry 180 specifies a cross-section “A4C” for the thumbnail image 611 selected by the user. The display of the thumbnail image 611 selected by the user is emphasized in dark gray.

The medical image display region 620 is divided into four segmented regions: a segmented region 621, a segmented region 622, a segmented region 623, and a segmented region 624. The segmented region 621 is set, for example, such that a medical image of the cross-section “A4C” is displayed. In the example shown in FIG. 6, since the thumbnail image 611 selected by the user represents the cross-section “A4C,” a medical image corresponding to the thumbnail image 611 is displayed in the segmented region 621.

Also, for example, the segmented region 622 is set such that a medical image of a cross-section “A2C” is displayed, the segmented region 623 is set such that a medical image of a cross-section “A3C” is displayed, and the segmented region 624 is set such that the result of the analysis of the GLS (bullseye figure) is displayed. In the example shown in FIG. 6, since only the cross-section “A4C” is set, nothing is displayed in the segmented region 622 and the segmented region 623, and only a template of a bullseye figure is displayed in the segmented region 624.

### (Step ST113)

After specifying the first cross-section, the processing circuitry 180 obtains supplementary information relating to the first low-resolution image. The supplementary information is, for example, the time at which the first medical image data is acquired, the heart rate, and the type of probe. After step ST113, the user-selected-cross-section process is completed, and the process proceeds to step ST120.

### (Step ST120)

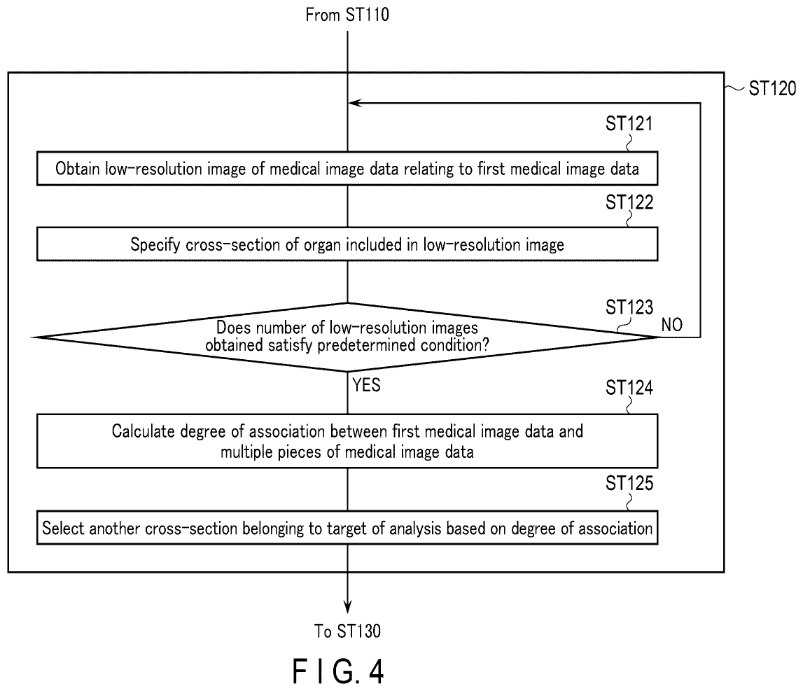

The processing circuitry 180 performs the another-cross-section selecting process. Hereinafter, a specific example of the another-cross-section selecting process will be explained with reference to the flowchart shown in FIG. 4. The another-cross-section selecting process may be referred to as “a first-stage determining process.”

FIG. 4 is a flowchart illustrating the another-cross-section selecting process shown in FIG. 2. The flowchart shown in FIG. 4 transitions from step ST110 shown in FIG. 2.

### (Step ST121)

When the processing circuitry 180 performs the another-cross-section selecting process, the processing circuitry 180 obtains a low-resolution image of medical image data related to the first medical image data with the obtaining function 184. At this time, the processing circuitry 180 may search for medical image data in order of closeness of the time of the acquisition to the time at which the first medical image data is acquired. Thus, the processing circuitry 180 can obtain a low-resolution image of medical image data in order of closeness of the time of the acquisition to the time at which the first medical image data is acquired. The processing circuitry 180 may also obtain a low-resolution image of medical image data acquired in the same mode as the mode (e.g., “2D single” mode) of the ultrasound diagnostic apparatus that acquires the first medical image data. Thus, the processing circuitry 180 can exclude the medical image data acquired in a mode different from the mode of the targets of analysis.

### (Step ST122)

After obtaining the low-resolution image, the processing circuitry 180 specifies a cross-section of an organ included in the low-resolution image with the specifying function 185.

### (Step ST123)

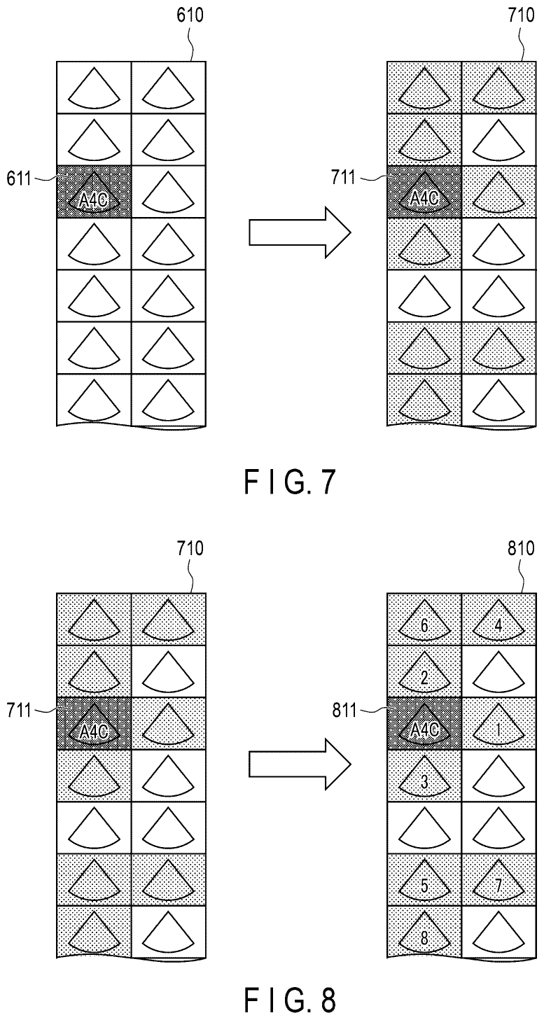

After specifying a cross-section of an organ, the processing circuitry 180 determines whether or not the number of low-resolution images obtained satisfies a predetermined condition. The predetermined condition is, for example, 10 pieces of medical image data for each of before and after the time at which the first medical image data selected by the user is acquired (i.e., 20 pieces of medical image data in total), that is, the number of low-resolution images of medical image data. If the number of pieces of medical image data is less than 20, the predetermined condition is the number of existing pieces of medical image data. If it is determined that the number of low-resolution images obtained does not satisfy the predetermined condition, the process returns to step ST121. On the other hand, if it is determined that the number of low-resolution images obtained satisfies the predetermined condition, the process proceeds to step ST124. A state of a thumbnail image display region after the number of low-resolution images obtained satisfies the predetermined condition will be explained below with reference to FIG. 7. In FIG. 7, only 14 small regions with thumbnail images are extracted from the thumbnail image display region and shown. The same applies to the subsequent figures, FIGS. 8, 9, 10, 12, and 13.

FIG. 7 is a diagram for explaining a state of a thumbnail image display region in which thumbnail images as candidates for analysis are displayed in a manner highlighted in light gray according to the first embodiment. FIG. 7 shows the thumbnail image display region 610 and a thumbnail image display region 710 as of a transition from step ST123 to step ST124. In addition to a thumbnail image 711 selected by a user, eight thumbnail images as candidates for analysis are displayed in the thumbnail image display region 710 in a manner highlighted in light gray. As of the time when the eight thumbnail images are displayed in a highlighted manner (i.e., as of the transition from step ST123 to step ST124), cross-sections related to the respective thumbnail images are specified; however, for convenience of explanation, display of the names of the cross-sections on the thumbnail images is omitted.

### (Step ST124)

After determining that the number of low-resolution images obtained satisfies the predetermined condition in step ST123, the processing circuitry 180 implements the calculating function 186. With the calculating function 186, the processing circuitry 180 calculates a degree of association between the first medical image data and each piece of medical image data. Specifically, the processing circuitry 180 calculates the degree of association by using supplementary information of each piece of medical image data. A state of a thumbnail image display region after calculating the degree of association will be explained below with reference to FIG. 8.

FIG. 8 is a diagram for explaining a state of a thumbnail image display region in which an order of analysis is displayed on thumbnail images as candidates for analysis according to the first embodiment. FIG. 8 shows the thumbnail image display region 710 and a thumbnail image display region 810 after calculating the degree of association. Numbers are displayed on the eight thumbnail images displayed in a highlighted manner in the thumbnail image display region 810 to indicate the order of the analysis based on the time of the acquisition of the degree of association. These numbers are determined based on the time interval between the time at which each thumbnail image is acquired and the time at which a thumbnail image 811 selected by a user is acquired.

### (Step ST125)

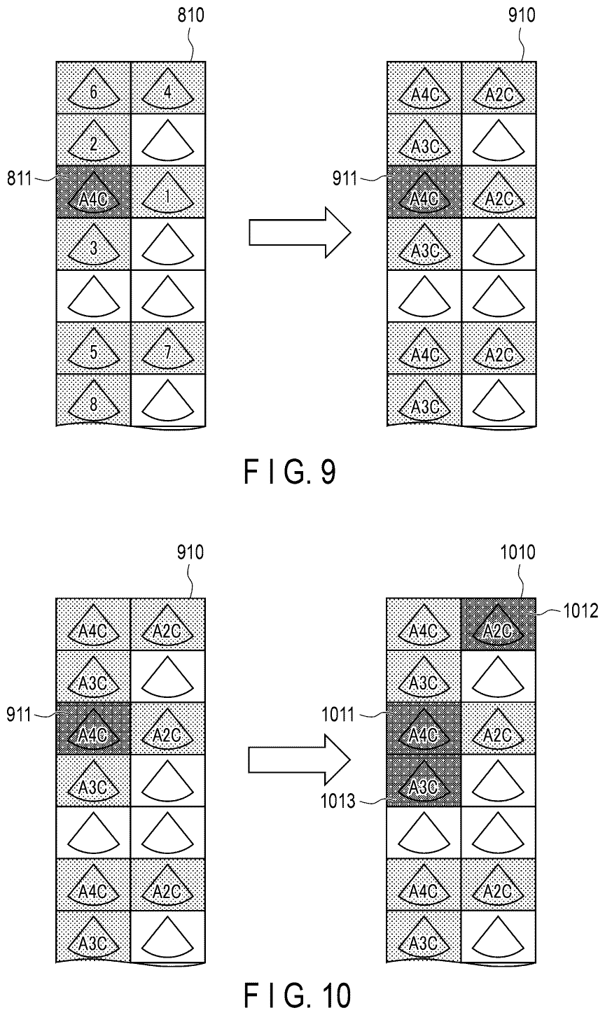

After calculating the degree of association, the processing circuitry 180 implements the selecting function 187. When the processing circuitry 180 implements the selecting function 187, the processing circuitry 180 selects another cross-section belonging to the target of analysis based on the degree of association. Specifically, the processing circuitry 180 performs the selecting process in order of closeness of the time at which the multiple pieces of medical image data are acquired to the time at which the selected medical image data is acquired (i.e., in the above-mentioned order of analysis). In the selecting process, another cross-section to be selected is based on a group of targets of analysis associated with the myocardial function analysis application. That is, the processing circuitry 180 selects a target of analysis different from the target of analysis selected by the user based on the group of targets of analysis. After step ST125, the another-cross-section selecting process is completed, and the process proceeds to step ST130. A state of a thumbnail image display region in the middle of the selecting process will be explained below with reference to FIG. 9.

FIG. 9 is a diagram for explaining a state of a thumbnail image display region in which the names of cross-sections are displayed on thumbnail images as candidates for analysis according to the first embodiment. FIG. 9 shows the thumbnail image display region 810 and a thumbnail image display region 910 after displaying the names. In the thumbnail image display region 910, the names of cross-sections are displayed on the eight thumbnail images that are displayed in a highlighted manner. At this time, since a thumbnail image 911 selected by a user is the cross-section “A4C,” other cross-sections belonging to the target of analysis are the cross-sections “A2C” and “A3C.” Next, a state of a thumbnail image display region after selecting a target of analysis will be explained with reference to FIG. 10.

FIG. 10 is a diagram for explaining a state of a thumbnail image display region in which thumbnail images as targets of analysis are displayed in an emphasized manner according to the first embodiment. FIG. 10 shows the thumbnail image display region 910 and a thumbnail image display region 1010 after selecting the target of analysis. In addition to a thumbnail image 1011 selected by a user, two thumbnail images 1012 and 1013, among the eight thumbnail images displayed in a highlighted manner, are displayed as the targets of analysis in an emphasized manner in the thumbnail image display region 1010.

In the thumbnail image display region 1010 shown in FIG. 10, rather than a thumbnail image (cross-section “A2C”) that is the first closest to the time at which the thumbnail image 1011 (cross-section “A4C”) selected by the user is acquired, the thumbnail image 1012 (cross-section “A2C”) that is the fourth closest to the time at which the thumbnail image 1011 (cross-section “A4C”) selected by the user is acquired, is selected and displayed in an emphasized manner. This indicates that the medical image data of the fourth closest thumbnail image 1012 has a larger degree of association than the degree of association of the medical image data of the first closest thumbnail image.

Since all of above FIGS. 7 to 10 are explanatory diagrams, the display forms of the thumbnail image display regions shown in the respective figures may or may not be displayed on the display.

In the flowchart of FIG. 4, step ST122 and step ST123 may be shuffled. That is, step ST123 is performed after step ST121; if the result of the process is Yes in step ST123, step ST122 is performed; then, after step ST122, step ST124 is performed. With such an order of the procedure, the processing circuitry 180 can obtain multiple thumbnails generated in advance, and collectively specify cross-sections of an organ using the multiple thumbnails obtained.

### (Step ST130)

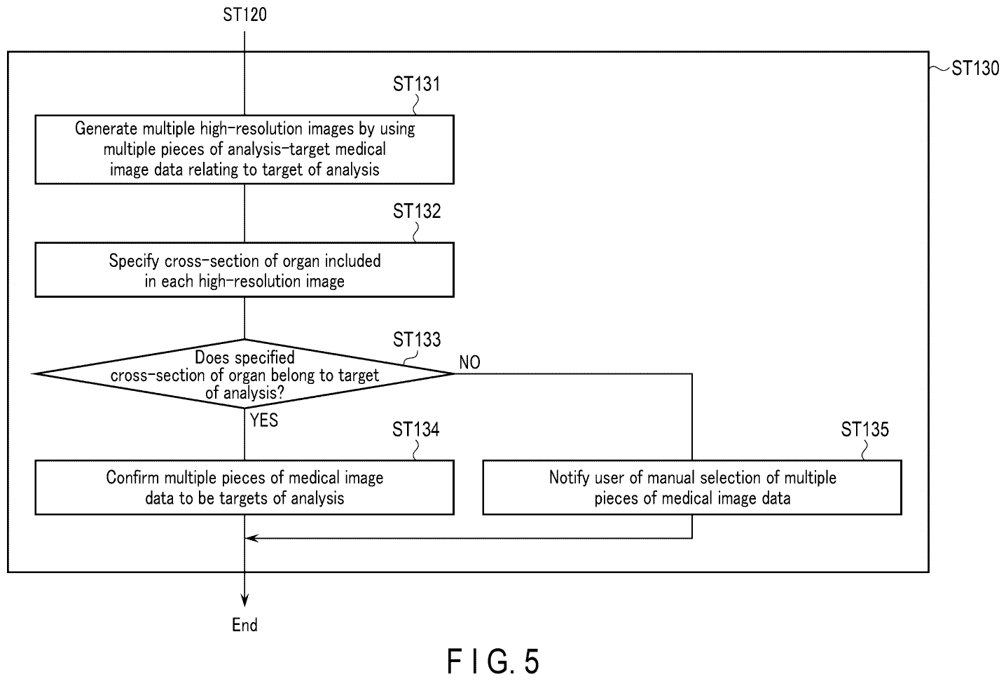

The processing circuitry 180 performs the selected-cross-section determining process. Hereinafter, a specific example of the selected-cross-section determining process will be explained with reference to the flowchart shown in FIG. 5. The selected-cross-section determining process may be referred to as “a second-stage determining process.”

FIG. 5 is a flowchart illustrating the selected-cross-section determining process shown in FIG. 2. The flowchart shown in FIG. 5 transitions from step ST120 shown in FIG. 2.

### (Step ST131)

When the processing circuitry 180 performs the selected-cross-section determining process, the processing circuitry 180 implements the image-generating function 183. When the processing circuitry 180 implements the image-generating function 183, the processing circuitry 180 generates multiple high-resolution images by using multiple pieces of analysis-target medical image data related to targets of analysis.

### (Step ST132)

After generating multiple high-resolution images, the processing circuitry 180 specifies a cross-section of an organ included in each of the high-resolution images with the specifying function 185. Specifying a cross-section of an organ is performed using low-resolution images in step ST122. In this step, a cross-section of an organ is specified again using high-resolution images.

### (Step ST133)

After specifying a cross-section of an organ, the processing circuitry 180 determines whether or not the specified cross-section of an organ belongs to the target of analysis. Whether or not the specified cross-section of an organ belongs to the target of analysis is based on the group of targets of analysis used in step ST125. If it is determined that the specified cross-section of an organ belongs to the target of analysis, the process proceeds to step ST134. On the other hand, if it is determined that the specified cross-section of an organ does not belong to the target of analysis or if the targets of analysis are overlapping, the process proceeds to step ST135.

### (Step ST134)

After determining that the specified cross-section of an organ belongs to the target of analysis in step ST133, the processing circuitry 180 confirms multiple pieces of medical image data to be targets of analysis. At this time, the processing circuitry 180 may associate the multiple pieces of medical image data confirmed to be targets of analysis with each other. After step ST134, the selected-cross-section determining process is completed, and the analysis-target extraction process shown in FIG. 2 is completed.

### (Step ST135)

After determining that the specified cross-section of an organ does not belong to the target of analysis in step ST133, the processing circuitry 180 notifies the user of manual selection of multiple pieces of medical image data. Specifically, the processing circuitry 180 presents a dialog indicating that manual selection is necessary to the user. The processing circuitry 180 may also present multiple cross-sections that may belong to the target of analysis to prompt the user to make a selection. After step ST135, the selected-cross-section determining process is completed, and the analysis-target extraction process shown in FIG. 2 is completed.

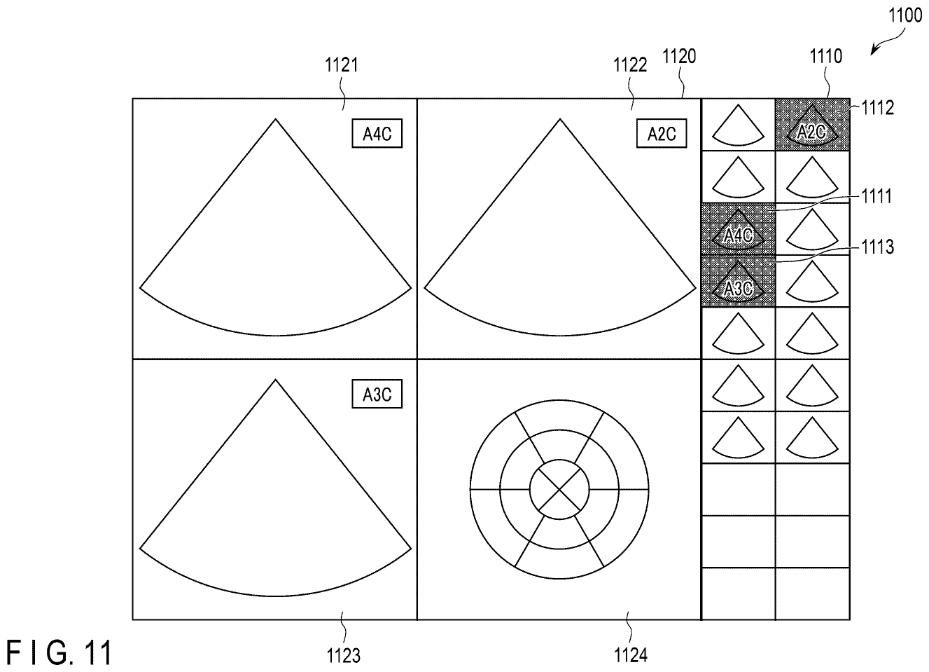

FIG. 11 is a diagram for illustrating a display screen 1100 posterior to the analysis of the myocardial function analysis application according to the first embodiment. The display screen 1100 shown in FIG. 11 includes a thumbnail image display region 1110 and a medical image display region 1120. The forms of the thumbnail image display region 1110 and the medical image display region 1120 are the same as the forms of the thumbnail image display region 610 and the medical image display region 620 shown in FIG. 6.

In the thumbnail image display region 1110, two thumbnail images 1112 (cross-section “A2C”) and 1113 (cross-section “A3C”) in addition to a thumbnail image 1111 (cross-section “A4C”) selected by a user are displayed as the targets of analysis in an emphasized manner.

In the medical image display region 1120, a segmented region 1121 displays a medical image corresponding to the thumbnail image 1111 (cross-section “A4C”). A segmented region 1122 displays a medical image corresponding to the thumbnail image 1112 (cross-section “A2C”), a segmented region 1123 displays a medical image corresponding to the thumbnail image 1113 (cross-section “A3C”), and a segmented region 1124 displays the result of the analysis of the GLS (bullseye figure).

To give an outline, if a user executes the myocardial function analysis application for determining the GLS and selects one thumbnail image (cross-section “A4C”) as a target of analysis, the processing circuitry 180 of the ultrasound diagnostic apparatus 1 selects the remaining two cross-sections (cross-sections “A2C” and “A3C”) as a group for determining the GLS. The processing circuitry 180 then not only causes those thumbnail images to be displayed in an emphasized manner but also causes the medical images of the respective cross-sections to be displayed and causes the result of the analysis of the GLS (bullseye figure) based on the respective medical images to be displayed. If a user selects a different thumbnail image on a display screen posterior to the analysis by the myocardial function analysis application, the processing circuitry 180 performs the above processes again, and updates the display screen to a display screen related to the selected different thumbnail image for display.

### First Modification of First Embodiment

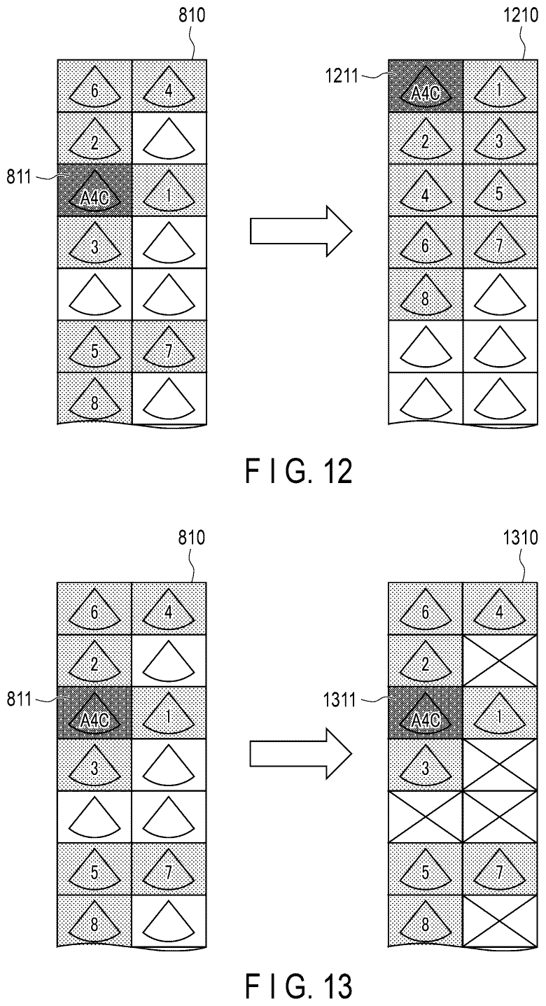

FIG. 12 is a diagram for explaining a state of a thumbnail image display region in which the candidates for analysis are rearranged according to a first modification of the first embodiment. FIG. 12 shows the thumbnail image display region 810 and a thumbnail image display region 1210 after rearranging the candidates for analysis.

The thumbnail image display region 1210 includes a thumbnail image 1211 displayed in an emphasized manner by being selected by a user, eight thumbnail images displayed in a highlighted manner as candidates for analysis, and the other thumbnail images. The thumbnail image 1211 is positioned on the upper left side of the thumbnail image display region 1210, and the eight thumbnail images are positioned in order of closeness of the time of the acquisition to the time at which the medical image data corresponding to the thumbnail image 1211 is acquired.

According to the first modification of the first embodiment, when a user manually selects medical image data, thumbnail images are rearranged in order of closeness of the time at which other pieces of medical image data are acquired to the time at which the medical image data first selected by the user is acquired. Since this allows the user to visually recognize the rearranged thumbnail images, the visibility of the thumbnail image display region can be improved.

### Second Modification of First Embodiment

FIG. 13 is a diagram for explaining a state of a thumbnail image display region in which thumbnail images other than the candidates for analysis are displayed as non-selected according to a second modification of the first embodiment. FIG. 13 shows the thumbnail image display region 810 and a thumbnail image display region 1310 after performing the non-selection display process.

The thumbnail image display region 1310 includes a thumbnail image 1311 displayed in an emphasized manner by being selected by a user and eight thumbnail images displayed in a highlighted manner as candidates for analysis. The thumbnail images other than the thumbnail image 1311 and the eight thumbnail images are displayed so as not to be selected, that is, displayed as non-selected, with a symbol “x” given thereto.

According to the second modification of the first embodiment, when a user manually selects medical image data, thumbnail images that need not be selected are displayed as non-selected. Since this eliminates the need for the user to check unnecessary thumbnail images, the visibility of the thumbnail image display region can be improved.

### Third Modification of First Embodiment

In the first embodiment, a series of processes starting from the execution of the myocardial function analysis application to the generation of the display screen illustrated in FIG. 11 is described. On the other hand, in a third modification of the first embodiment, a process after the generation of the display screen illustrated in FIG. 11 will be explained.

Specifically, in the third modification of the first embodiment, it is assumed that a user selects a different thumbnail image after the display screen illustrated in FIG. 11 is displayed. A setting can be made in advance by a user so that the processing circuitry 180, for example, performs either one of the following two processes (a first process and a second process) when a user selects a different thumbnail image on a display screen posterior to the analysis by the myocardial function analysis application.

In the first process, after a user selects a different thumbnail image, a different medical image is also selected based on the different thumbnail image selected. That is, the first process is the same as performing the flowchart of FIG. 2 again. In this case, the processing circuitry 180 performs an analyzing process based on a medical image included in a different target of analysis including a medical image corresponding to a different thumbnail image, and generates a different result of analysis.

In the second process, only the different thumbnail image selected by the user is changed. For example, if a user wishes to change the medical image of the cross-section “A3C” displayed in the segmented region 1123 on the display screen 1100 shown in FIG. 11, the user selects the thumbnail image of the cross-section “A3C” from the thumbnail image display region 1110. After the user selects the thumbnail image, the processing circuitry 180 changes the medical image included in the target of analysis to a medical image corresponding to the thumbnail image selected, performs an analysis process based on the medical image after the change, and generates a different result of analysis based on a different target of analysis.

According to the first process and the second process described above, the processing circuitry 180 may cause medical images in a different target of analysis including a different medical image selected by a user and a different result of analysis generated from the different target of analysis to be displayed. At this time, the medical images in the different target of analysis differ in at least one medical image from the medical images of the target of analysis.

According to the third modification of the first embodiment, a user can flexibly change the medical image for use in an analysis process even after the myocardial function analysis application is executed. Thus, user's convenience related to performing the myocardial function analysis process can be improved.

### Fourth Modification of First Embodiment

In the first embodiment, a user selects any thumbnail using the thumbnail image display region included in the display screen posterior to the execution of the myocardial function analysis application as shown in FIG. 6. On the other hand, in a fourth modification of the first embodiment, a user may select any thumbnail on a display screen (such as an examination-list screen) prior to the execution of the myocardial function analysis application.

The examination-list screen includes, for example, a thumbnail image display region related to an examination image of a patient captured on the day of the examination. A user may select any thumbnail using the thumbnail image display region included in the examination-list screen. After a user selects any thumbnail, the myocardial function analysis application may be executed by the user pressing a software button (e.g., a button for executing the myocardial function analysis application) that is being displayed (or has been displayed) on the examination-list screen. In this case, a thumbnail is selected at the time when the myocardial function analysis application is executed.

According to the fourth modification of the first embodiment, a user can select a thumbnail and perform the myocardial function analysis process using not only the display screen of the myocardial function analysis application but also any display screen. Thus, user's convenience related to performing the myocardial function analysis process can be improved.

### Another Specific Example of First Embodiment

In the first embodiment described above, a myocardial function analysis application for determining a GLS is explained as a specific example. In another specific example of the first embodiment, an application for performing stress echocardiographic examination (analysis) is explained.

In the stress echocardiographic analysis, the same cross-section is acquired as a target of analysis in a first phase before applying stress to a patient and a second phase after applying stress to a patient. The target of analysis is, for example, a group of cross-sections of a heart. In the case of a stress echocardiographic analysis process, the group of cross-sections of a heart is, for example, an apical two chamber view, an apical four chamber view, a parasternal long-axis view, and a parasternal short-axis view.

For example, in a stress echocardiographic analysis, the processing circuitry 180 may perform substantially the same process as in the first embodiment. There is phase information as information specific to stress echocardiographic analysis. The phase information is, for example, information indicating which of the first phase or the second phase the acquired medical image data belongs to. The phase information is, for example, attached to medical image data as supplementary information. Thus, with the calculating function 186, the processing circuitry 180 may calculate the degree of association between pieces of medical image data further based on the phase information of stress echocardiography attached to the medical image data. For example, the degree of association based on the phase information increases if the pieces of medical image data are in the same phase. In other words, the degree of association between pieces of medical image data that are in the same phase of stress echocardiographic analysis calculated by the processing circuitry 180 has a large value of degree of association.

As a specific use, the processing circuitry 180 can select medical image data targeted for analysis among the medical image data acquired at the time of the stress echocardiography and associate the multiple pieces of medical image data confirmed as targets of analysis for each phase. In addition, if medical image data reacquired has a higher degree of association than the degree of association of the medical image data already acquired, the processing circuitry 180 can also replace the medical image data targeted for analysis.

The processing circuitry 180 may have a stress echocardiographic analysis function (stress echocardiographic analyzer) for performing a stress echocardiographic analysis. With the stress echocardiographic analysis function, the processing circuitry 180 may generate a result of the stress echocardiographic analysis by analyzing a medical image data group related to a target of analysis. This medical image data group, for example, includes medical image data selected by a user.

To sum up, the ultrasound diagnostic apparatus according to the first embodiment searches for a thumbnail around a thumbnail selected by a user (a selected thumbnail) and thereby automatically selects a thumbnail that shows a cross-section of an organ other than the cross-section of the organ shown by the selected thumbnail. Specifically, the ultrasound diagnostic apparatus according to the first embodiment automatically selects a medical image from among multiple candidate medical images corresponding to automatically selected multiple thumbnails based on at least one of those having a close heart rate, those being in the same stress echo phase, or those acquired by the same probe. The ultrasound diagnostic apparatus according to the first embodiment generates a result of analysis based on a medical image selected by a user and a medical image automatically selected, and generates a display image that includes multiple medical images and the result of analysis. Also, if a different thumbnail is selected by a user after the display image is generated, the ultrasound diagnostic apparatus according to the first embodiment updates the result of analysis and the display image based on a medical image corresponding to the different thumbnail selected by the user.

As explained above, the ultrasound diagnostic apparatus according to the first embodiment obtains a low-resolution image (thumbnail) of each piece of cross-section image data related to first medical image data (first cross-section image data) selected by a user and showing a cross-section of an organ, specifies a cross-section of an organ included in each of the thumbnails by using the multiple thumbnails obtained, and selects a cross-section for use as a target of analysis other than the cross-section selected by the user from among the specified cross-sections based on a degree of association between the first cross-section image data and each piece of cross-section image data.

Therefore, the ultrasound diagnostic apparatus according to the first embodiment can efficiently select a cross-section suitable for a desired analysis by being able to specify a cross-section from a low-resolution image (thumbnail) and select a cross-section belonging to the target of analysis based on the degree of association.

### Second Embodiment

In the first embodiment, an ultrasound diagnostic apparatus that performs a myocardial function analysis process is described. On the other hand, in a second embodiment, an analysis apparatus that performs a myocardial function analysis process will be described.

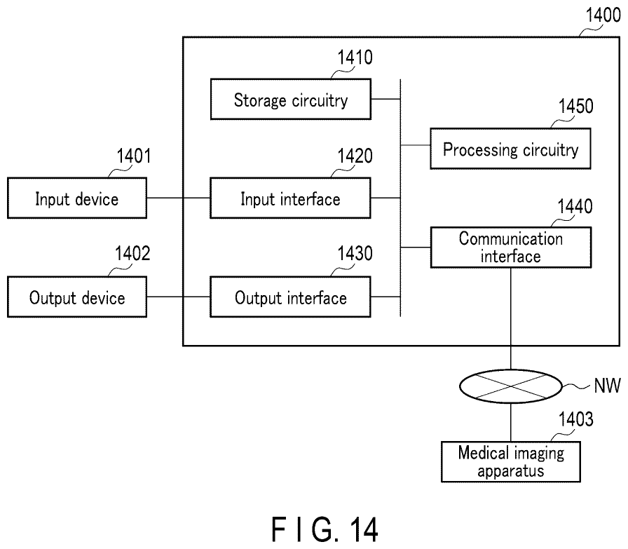

FIG. 14 is a block diagram showing an example of a configuration of an analysis apparatus 1400 according to the second embodiment. The analysis apparatus 1400 shown in FIG. 14 is connected to an input device 1401 and an output device 1402. The analysis apparatus 1400 is also connected to a medical imaging apparatus 1403 via a network NW. The medical imaging apparatus 1403 corresponds to, for example, an ultrasound diagnostic apparatus. The input device 1401, which is substantially the same as the input device 102 shown in FIG. 1, typically corresponds to a mouse and a keyboard. The output device 1402 is substantially the same as the output device 103 shown in FIG. 1.

The analysis apparatus 1400 is, for example, a computer capable of executing a myocardial function analysis application. The analysis apparatus 1400 includes storage circuitry 1410, an input interface 1420, an output interface 1430, a communication interface 1440, and processing circuitry 1450.

The storage circuitry 1410 includes, for example, a processor-readable storage medium, such as a magnetic storage medium, an optical storage medium, or a semiconductor memory. The storage circuitry 1410 stores a program related to myocardial function analysis, various kinds of data, and the like. The various kinds of data include, for example, parameters and LUTs that are used during execution of a program. For example, the programs and various kinds of data may be pre-stored in the storage circuitry 1410. For example, the programs and various kinds of data may be stored in a non-transitory storage medium, distributed, read from the non-transitory storage medium, and installed in the storage circuitry 1410. In accordance with an operation input via the input interface 1420, the storage circuitry 1410 stores medical image data generated by the medical imaging apparatus 1403, and the like. The storage circuitry 1410 can also transfer the stored medical image data to an external device, etc., via the communication interface 1440.

The storage circuitry 1410 may be a drive device or the like which reads and writes various kinds of information to and from a portable storage medium such as a CD drive, a DVD drive, and a flash memory. The storage circuitry 1410 can also write the stored data into a portable storage medium, and store the data in an external device via the portable storage medium.

The input interface 1420 receives various instructions from an operator via the input device 1401. The input interface 1420 is connected to the processing circuitry 1450 via a bus, for example, converts an operation instruction that is input by an operator into an electrical signal, and outputs the electrical signal to the processing circuitry 1450. The input interface 1420 is not limited to a component that is connected to physical operational components such as a mouse and a keyboard. Examples of the input interface also include circuitry that receives an electrical signal corresponding to an operation instruction that is input from an external input device provided separately from the analysis apparatus 1400 and outputs the electrical signal to the processing circuitry 1450.

The output interface 1430 is, for example, an interface for outputting an electrical signal from the processing circuitry 1450 to the output device 1402. The output interface 1430 is connected to the processing circuitry 1450 via a bus, for example, and outputs an electrical signal from the processing circuitry 1450 to the output device 1402.

The communication interface 1440 is connected to the medical imaging apparatus 1403 and an external apparatus via a network NW, for example, and performs data communication with each of the apparatuses.

The processing circuitry 1450 is, for example, a processor that functions as the center of the analysis apparatus 1400. The processing circuitry 1450 executes a program stored in the storage circuitry 1410, thereby implementing a function corresponding to the program. The processing circuitry 1450 includes the obtaining function 184 (obtaining unit), the specifying function 185 (specifying unit), the calculating function 186 (calculating unit), the selecting function 187 (selecting unit), the display control function 188 (display controller), and the system control function 189 (controller) of the first embodiment. Since these various functions are substantially the same as those of the first embodiment, descriptions thereof will be omitted.

As explained above, the analysis apparatus according to the second embodiment obtains a low-resolution image (thumbnail) of each piece of cross-section image data related to first medical image data (first cross-section image data) selected by a user and showing a cross-section of an organ, specifies a cross-section of an organ included in each of the thumbnails by using the multiple thumbnails obtained, and selects a cross-section for use as a target of analysis other than the cross-section selected by the user from among the specified cross-sections based on a degree of association between the first cross-section image data and each piece of cross-section image data.

Therefore, advantageous effects similar to those of the first embodiment can be expected from the analysis apparatus according to the second embodiment.

According to at least one embodiment described above, it is possible to efficiently select a cross-section suitable for a desired analysis.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An analysis apparatus comprising processing circuitry configured to:
obtain a thumbnail of each of multiple pieces of cross-section image data related to first cross-section image data selected by a user and showing a cross-section of an organ;
specify, using the multiple thumbnails obtained, a cross-section of an organ included in each of the multiple thumbnails; and
select a cross-section for use as a target of analysis other than a cross-section selected by the user from the specified cross-sections based on a degree of association between the first cross-section image data and each of the multiple pieces of cross-section image data.

2. The analysis apparatus according to claim 1, wherein the thumbnail is information regarding the cross-section of the organ.

3. The analysis apparatus according to claim 1, wherein the processing circuitry is further configured to obtain the multiple thumbnails of the multiple pieces of cross-section image data acquired in a same mode as a mode of an ultrasound diagnostic apparatus that acquires the first cross-section image data.

4. The analysis apparatus according to claim 1, wherein the processing circuitry is further configured to search for the multiple thumbnails in order of closeness of a time at which each of the multiple pieces of cross-section image data is acquired to a time at which the first cross-section image data is acquired.

5. The analysis apparatus according to claim 1, wherein the degree of association is based on a time at which the first cross-section image data is acquired.

6. The analysis apparatus according to claim 5, wherein the degree of association is based on at least one of a heart rate, a type of probe, or phase information of stress echocardiography attached to the first cross-section image data.

7. The analysis apparatus according to claim 1, wherein the processing circuitry is further configured to calculate the degree of association between the first cross-section image data and each of the multiple pieces of cross-section image data.

8. The analysis apparatus according to claim 1, wherein the target of analysis is a group of cross-sections of a heart.

9. The analysis apparatus according to claim 8, wherein the group includes an apical two chamber view, an apical three chamber view, and an apical four chamber view, or an apical two chamber view, an apical four chamber view, a parasternal long-axis view, and a parasternal short-axis view.

10. The analysis apparatus according to claim 1, wherein the processing circuitry is further configured to display the multiple thumbnails in such a manner that the multiple thumbnails are rearranged in order of closeness of a time at which each of the multiple pieces of cross-section image data is acquired to a time at which the first cross-section image data is acquired.

11. The analysis apparatus according to claim 1, wherein the processing circuitry is further configured to display the multiple thumbnails in such a manner that a form of display is changed according to whether they are cross-section image data related to the target of analysis.

12. The analysis apparatus according to claim 1, wherein the processing circuitry is further configured to
generate a result of analysis by analyzing cross-section image data in the target of analysis including the first cross-section image data, and
display the cross-section image data in the target of analysis and the result of analysis.

13. The analysis apparatus according to claim 1, wherein the processing circuitry is further configured to display cross-section image data in a different target of analysis including different cross-section image data selected by the user and a different result of analysis generated from the different target of analysis, wherein the cross-section image data in the different target of analysis differs from cross-section image data in the target of analysis in at least one piece of cross-section image data.

14. The ultrasound diagnostic apparatus comprising processing circuitry configured to:
obtain a thumbnail of each of multiple pieces of cross-section image data related to first cross-section image data selected by a user and showing a cross-section of an organ;
specify, using the multiple thumbnails obtained, a cross-section of an organ included in each of the multiple thumbnails; and
select a cross-section for use as a target of analysis other than a cross-section selected by the user from the specified cross-sections based on a degree of association between the first cross-section image data and each of the multiple pieces of cross-section image data.

15. A non-transitory computer-readable storage medium storing a program for causing a computer to execute processing comprising:
obtaining a thumbnail of each of multiple pieces of cross-section image data related to first cross-section image data selected by a user and showing a cross-section of an organ;
specifying, using the multiple thumbnails obtained, a cross-section of an organ included in each of the multiple thumbnails; and
selecting a cross-section for use as a target of analysis other than a cross-section selected by the user from the specified cross-sections based on a degree of association between the first cross-section image data and each of the multiple pieces of cross-section image data.
